(19) United States
(12) Patent Application Publication
(10) Pub. No.: US 2003/0229267 A1
(43) Pub. Date: Dec. 11, 2003

(54) Obstetrical imaging system and integrated fetal vacuum extraction system

(76) Inventors: Amir Belson, Cupertino, CA (US); Doron Kreiser, Herzlia (IL)

Correspondence Address: LEARY & ASSOCIATES, 3900 NEWPARK MALL RD., THIRD FLOOR, SUITE 317, NEWARK, CA, 94560, US

(21) Appl. No.: 10/138,175

(22) Filed: May 3, 2002

Related U.S. Application Data
(63) Continuation-in-part of application No. 10/024,656, filed on Dec. 17, 2001.
(60) Provisional application No. 60/256,155, filed on Dec. 15, 2000.

(51) Int. Cl. A61B 1/04

(52) U.S. Cl. 600/109

(57) ABSTRACT

A finger-mounted obstetrical imaging system is described for augmenting digital pelvic examination and other obstetrical procedures with videoendoscopic visualization. An obstetrical imaging system integrated with a fetal vacuum extraction instrument is described using tissue transillumination to assist in proper placement of the vacuum extraction instrument on the head of a fetus during delivery.

### CROSS REFERENCE TO OTHER APPLICATIONS

This application is a continuation-in-part of U.S. patent application Ser. No. 10/024,656, filed on Dec. 17, 2001, which claims the benefit of U.S. Provisional Patent Application, serial No. 60/256,155, filed on Dec. 15, 2000.

### FIELD OF THE INVENTION

The present invention relates generally to a medical imaging system. More particularly, it relates to an obstetrical imaging system and a vacuum fetal extraction instrument for assisting in childbirth.

### BACKGROUND OF THE INVENTION

Adequate diagnosis and intervention during labor are crucial to reduce maternal and fetal morbidity and mortality that are inherent risks of vaginal deliveries. Modern obstetrics is still considered to be an “art” instead of a “science”, as it is based on the operators' subjective impression and experience rather than analysis of objective clinical findings. The evaluation of the labor process is based on interpretation of digital pelvic examination. Moreover, residents who are still acquiring their skills perform most of the public deliveries in the U.S. Regardless, even the most experienced operators have up to thirty percent error rate.

Today, unlike other medical fields, there is no objective tool in the hands of obstetricians to assist with diagnosis and with the decision making process. Even the ultrasound, which is an important tool in obstetrics and gynecology, is not effective during labor since the pelvic bones create an acoustic shadow and prevent accurate imaging.

For evaluation of the labor and delivery processes, obstetricians must know the fetal lie, presentation and position. While the first two can be evaluated early in labor and usually are not changing, the fetal position, which is the position of the fetal occiput in relation to the maternal spine, is changing during labor and delivery. The diagnosis of the fetal position is done by feeling the fetal scalp sutures and fontanelles, which often cannot be sensed due to scalp edema or insufficient cervical dilation and mainly due to operator error. Another important factor in normal labor and delivery is the fetal weight. While the fetal weight can be evaluated quite accurately by ultrasound before the engagement of the head in the mother's pelvis, an ultrasonic evaluation of the fetal weight is not accurate during labor. Moreover, fetal weight evaluation by physical examination has an accuracy of ±1 kg. Erroneous evaluation of the fetal weight can cause unnecessary prolonged trail of labor that can result in cesarean section or shoulder dystocia. The prediction of the labor outcome depends on both the fetal position and the fetal weight. An early diagnosis of pathologic labor outcome leads to an early decision to perform a cesarean section that can improve fetal and maternal outcome.

Ten to thirty percent of all vaginal deliveries end in instrument delivery, mostly by vacuum extraction. Many of these procedures are performed in urgent fashion because of fetal distress. Again, a proper application of the vacuum extractor or forceps is based on the position of the fetal head and the location of the fontanelles. Malposition of the vacuum extractor or forceps increases the risk of failure that put the mother and the baby in higher risk. The fetal weight plays a major role in the chance to have a successful and safe delivery. Larger babies usually have longer deliveries and higher probability to have complications. An accurate method of diagnosis of the fetal weight during labor and delivery is unavailable today.

Fetal monitoring during labor and delivery is done by subjective interpretation of the fetal heat rate pattern. Frequently, the data is hard to evaluate and as a result, a fetal scalp blood sample must be obtained for blood pH testing. The fetal scalp blood sampling is a challenging process and the operator must have experience since the procedure demands direct visualization of the scalp.

One sign of fetal stress during labor is the presence of meconium in the amniotic fluid. Meconium can be detected in the amniotic fluid by direct visualization using an amnioscope inserted transcervically. Transcervical amnioscopy may not be possible without sufficient dilation of the cervix.

About three percent of the deliveries in the U.S. are twin deliveries. Delivery of the second twin is a technically difficult procedure, especially if the presenting part is not the head. Again, the fetal extraction is done after recognition of fetal parts by the operator through the intact membranes.

Normal delivery depends on the progression of the proceeding fetal part through the birth canal. There are pathologic situations in which there is a presenting umbilical cord, fetal blood vessel (vasa previa) or placental part (placenta previa) that can be compressed or ruptured and result in bleeding or fetal asphyxia. The diagnosis of these pathologies is done by physical examination or ultrasound and is not sufficient, which puts the mother and fetus in increased risk of morbidity and mortality. An accurate diagnosis can prevent these complications.

The risks of the mother during labor and delivery do not end with the expulsion of the baby. Postpartum complications are caused by rupture of the uterus and more commonly by retained parts of the placenta in the uterine cavity. The common method to diagnose these complications is by insertion of the physician's hand into the uterine cavity and a manual exploration for the uterine wall defect or the retained parts of the placenta. These procedures demand high skill and often is done by two operators after the failure of the first one to obtain the diagnosis.

In summary, “modern” obstetrics is still based on methods that have been in use for over a century. There is no use of modern technology in diagnosis and management of most complications during labor and delivery. A direct view of the birth canal can give objective information that can lead to more rapid and efficient management and interventions.

### SUMMARY OF THE INVENTION

In keeping with the foregoing discussion, in a first embodiment, the present invention provides a finger-mounted obstetrical imaging system for augmenting a digital pelvic examination with videoendoscopic visualization. The obstetrical imaging system includes a miniaturized video camera mounted on the obstetrician's finger or elsewhere on the hand and an illumination subsystem. The miniaturized video camera may be a charged couple device (CCD) camera, which is connected by way of a video cable to a display monitor. Alternatively, image signals from the miniaturized video camera may be transmitted to the display monitor by a wireless transmitter. The illumination subsystem includes a light source and a fiber optic cable that extends from the light source to the miniaturized video camera. Alternatively, the illumination subsystem may consist of a miniaturized light source mounted adjacent to the miniaturized video camera. The optical fibers within the fiber optic cable are arranged to direct light in front of the miniaturized video camera. The miniaturized video camera and distal ends of the optical fibers may be mounted on a ring that attaches to the obstetrician's finger. The miniaturized video camera and the optical fibers are preferably arranged to create a low profile configuration that is smoothly tapered on the proximal and distal ends to facilitate insertion and removal with the obstetrician's hand during a digital pelvic examination. The distal ends of the optical fibers may be arranged in a crescent that partially encircles the miniaturized video camera to provide a low profile and for effective illumination that enhances depth perception with the miniaturized video camera. Alternatively, the miniaturized video camera and the optical fibers can be integrated into a surgical glove.

In another embodiment, the present invention provides an obstetrical imaging system integrated with a vacuum extractor. The vacuum extractor includes a flexible or rigid vacuum-gripping cup, a vacuum pump connected to the cup via a vacuum hose and a pull handle attached to the cup. Optionally, the vacuum pump may be integrated into the pull handle of the device, as shown. An obstetrical imaging system is integrated into the vacuum extractor to facilitate correct placement of the vacuum-gripping cup on the head of the fetus. The obstetrical imaging system includes a miniaturized video camera mounted on the vacuum-gripping cup and an illumination subsystem. The miniaturized video camera may be permanently mounted on the vacuum-gripping cup or it may be removable so that the same the miniaturized video camera can be used interchangeably with the vacuum extractor and with the finger-mounted obstetrical imaging system. A video cable or wireless transmitter connects the miniaturized video camera to a display monitor. The illumination subsystem includes a light source and a fiber optic cable that is configured to distribute the illumination around the rim of the vacuum-gripping cup. In one particularly preferred embodiment, one or more optical fibers form a light-emitting ring that encircles the rim of the vacuum-gripping cup. The light from the light-emitting ring transilluminates the tissue of the scalp and makes the sutures, fontanelles and other structures beneath the scalp visible. Various configurations and features of the integrated obstetrical imaging system and integrated vacuum extractor are described.

The integrated videoendoscopic obstetrical imaging system facilitates safe placement of the vacuum extractor on the preferred target area on the head of the fetus, which is on the flexion point of the skull between the anterior and posterior fontanelles. The present invention also provides various other safety mechanisms for monitoring and/or limiting the pressure or force applied to the fetus by the vacuum extractor.

### BRIEF DESCRIPTION OF THE DRAWINGS

FIG. 1 shows a finger-mounted obstetrical imaging system for augmenting a digital pelvic examination with videoendoscopic visualization.

FIG. 2 is an enlarged detail view of the finger-mounted obstetrical imaging system of FIG. 1.

FIG. 3 shows a prior art vacuum extractor.

FIGS. 4a-4d show the preferred target area for placement of a vacuum extractor on a fetus.

FIG. 5 shows an obstetrical imaging system integrated with a vacuum extractor.

FIG. 6 is an underside view of the integrated obstetrical imaging system and vacuum extractor of FIG. 5.

FIG. 7 shows a cross section of the fiber optic light-emitting ring of the integrated obstetrical imaging system and vacuum extractor shown in FIG. 5.

FIGS. 8a-8d show the integrated obstetrical imaging system and vacuum extractor of FIG. 5 placed on a fetus.

FIG. 9 shows an alternate embodiment of the obstetrical imaging system integrated with a vacuum extractor.

FIG. 10 is an underside view of the integrated obstetrical imaging system and vacuum extractor of FIG. 9.

FIG. 11 shows an alternate embodiment of the integrated obstetrical imaging system and vacuum extractor.

FIG. 12 shows an alternate handle assembly for an integrated obstetrical imaging system and vacuum extractor.

FIG. 13 shows an alternate configuration of a vacuum extractor.

FIG. 14 shows the vacuum extractor of FIG. 13 placed on a fetus.

FIG. 15 shows an alternate configuration of a vacuum extractor.

FIG. 16 shows the vacuum extractor of FIG. 15 placed on a fetus.

FIG. 17 shows an alternate configuration of a vacuum extractor.

FIG. 18 shows the vacuum extractor of FIG. 17 placed on a fetus.

FIG. 19 shows an alternate configuration of a vacuum extractor.

FIG. 20 shows the vacuum extractor of FIG. 19 placed on a fetus.

FIG. 21 shows a vacuum extractor with an analog tension meter integrated into the pull handle.

FIG. 22 shows a vacuum extractor handle with an integral digital tension meter.

FIG. 23 shows a vacuum extractor handle with an adjustable tension drag.

FIG. 24 shows a miniaturized obstetrical imaging system integrated with a transcervical amnioscope.

FIG. 25 shows another alternate embodiment of the obstetrical imaging system integrated with a vacuum extractor.

FIG. 26 shows an embodiment of the integrated obstetrical imaging system and vacuum extractor including an array of contact or pressure sensors distributed around the rim of the vacuum-gripping cup to detect impending detachment of the vacuum extraction device.

FIG. 27 shows a monitor screen displaying information from an array of contact or pressure sensors distributed around the rim of the vacuum-gripping cup to indicate impending detachment of the vacuum extraction device.

### DETAILED DESCRIPTION OF THE INVENTION

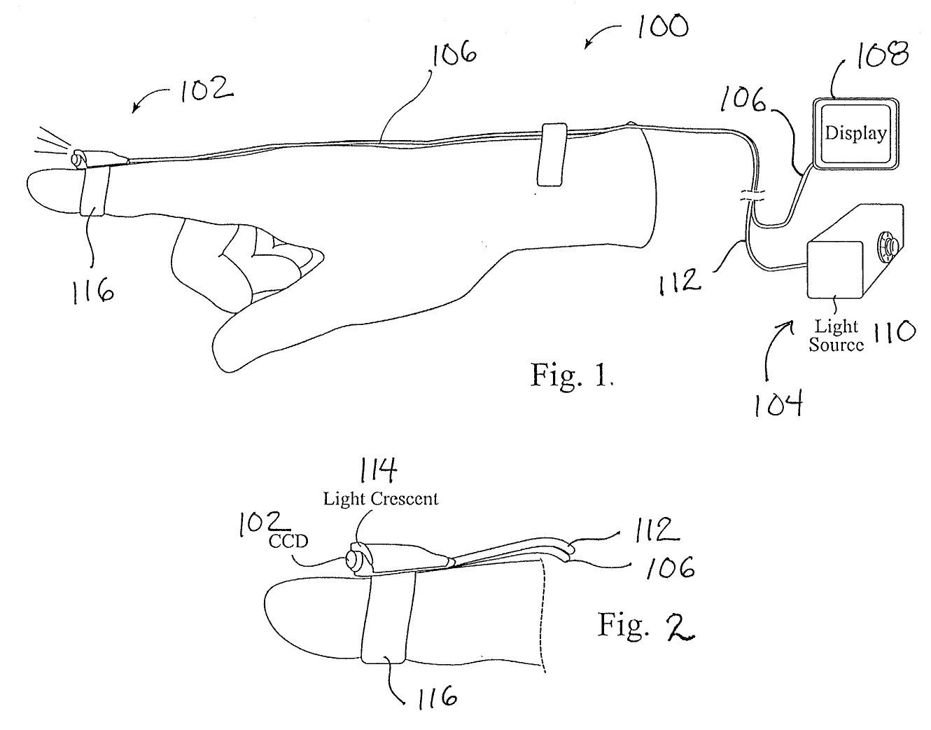

FIG. 1 shows a finger-mounted obstetrical imaging system 100 for augmenting a digital pelvic examination with videoendoscopic visualization. The obstetrical imaging system 100 includes a miniaturized video camera 102 mounted on the obstetrician's finger or elsewhere on the hand and an illumination subsystem 104. The miniaturized video camera 102 may be a charged couple device (CCD) camera, which is connected by way of a video cable 106 to a display monitor 108. Alternatively, image signals from the miniaturized video camera 102 may be transmitted to the display monitor 108 by a wireless transmitter. The illumination subsystem 104 includes a light source 110 and a fiber optic cable 112 that extends from the light source 110 to the miniaturized video camera 102. Alternatively, the illumination subsystem 104 may consist of a miniaturized light source mounted adjacent to the miniaturized video camera 102. The optical fibers 114 within the fiber optic cable 112 are arranged to direct light in front of the miniaturized video camera 102. In a preferred embodiment, the miniaturized video camera 102 and distal ends of the optical fibers 114 are mounted on a ring 116 that attaches to the obstetrician's finger, as shown in an enlarged detail drawing in FIG. 2. The miniaturized video camera 102 and the optical fibers 114 are preferably arranged to create a low profile configuration that is smoothly tapered on the proximal and distal ends to facilitate insertion and removal with the obstetrician's hand during a digital pelvic examination. In one particularly preferred embodiment, the distal ends of the optical fibers 114 are arranged in a crescent that partially encircles the miniaturized video camera 102 to provide a low profile and for effective illumination that enhances depth perception with the miniaturized video camera 102. Alternatively, the miniaturized video camera 102 and the optical fibers 114 can be integrated into a surgical glove.

Prior to performing a digital pelvic examination, the ring-mounted miniaturized video camera 102 is placed on the obstetrician's hand. The miniaturized video camera 102 is inserted into the patient to enable visualization of the vaginal vault, the cervical canal or the proceeding parts of the fetus. The miniaturized video camera 102 and the optical fibers 114 can be the presenting parts of the device or can be connected to the base of a miniaturized cup-like structure that will be positioned against the fetal scalp and by transillumination or reflection will give a digital image of the internal and/or external anatomy of the fetal scalp. The image from the miniaturized video camera 102 assists the obstetrician in diagnosing the position of the fetus and other conditions, as described above.

The obstetrical imaging system may utilize a full-size video display monitor 108, which may be mounted on a mobile cart for convenient positioning. Alternatively or in addition, the obstetrical imaging system 100 may include a small video display monitor 108 mounted on the physician's wrist and/or a head-mounted video display device to facilitate direct viewing and videoendoscopic viewing simultaneously. The video display monitor 108 may also be adapted to display additional data, such as fetal heart rate, fetal blood pH, etc.

In an alternate embodiment of the finger-mounted obstetrical imaging system, the miniaturized video camera 102 may be replaced with a fiber optic imaging bundle that is mounted on the physician's finger and a video camera 102 positioned at a location that remains external to the patient. This embodiment allows a very low profile imaging system without the need for miniaturized electronics.

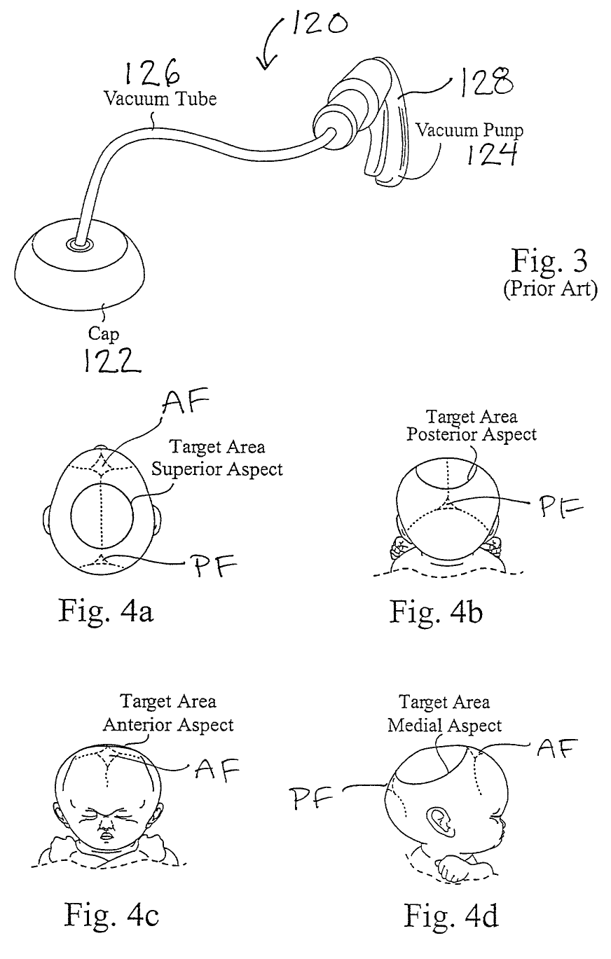

FIG. 3 shows a prior art vacuum extractor 120. Vacuum extractors 120 come in a variety of configurations, but generally they include a vacuum-gripping cup 122, a vacuum pump 124 connected to the cup 122 via a vacuum hose 126 and a pull handle 128 attached to the cup 122. The vacuum-gripping cup 122 may be flexible or rigid and is typically about 5 cm in diameter. The vacuum extractor 120 may be used with an electric vacuum pump or with a manually operated vacuum pump 124. Alternatively, the vacuum extractor 120 may be used with a hospital vacuum source. In some vacuum extractors 120, such as the one shown in FIG. 3, the vacuum pump 124 is integrated into the pull handle 128. In use, the cup 122 of the vacuum extractor 120 is placed on the head of the fetus and a vacuum is applied. It is important to position the vacuum-gripping cup 122 correctly on the head of the fetus. FIGS. 4a-4d show the preferred target area for placement of the vacuum-gripping cup 122 on the head of the fetus, which is on the flexion point of the skull between the anterior fontanelle AF and posterior fontanelle PF. Since in the majority of cases where a vacuum extractor 120 is needed, the fetus is not visible to the obstetrician, the correct position for the vacuum-gripping cup 122 must be estimated based on palpation of the fetal scalp to identify the anterior fontanelle AF and posterior fontanelle PF. This can be difficult under the best of circumstances, but may be impossible when scalp edema is present.

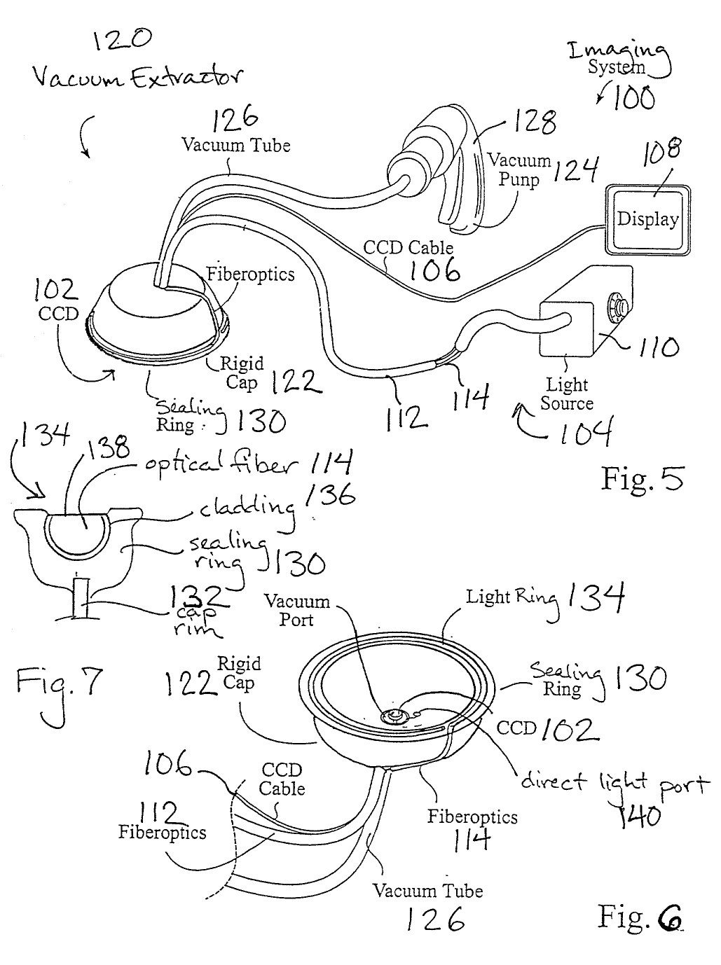

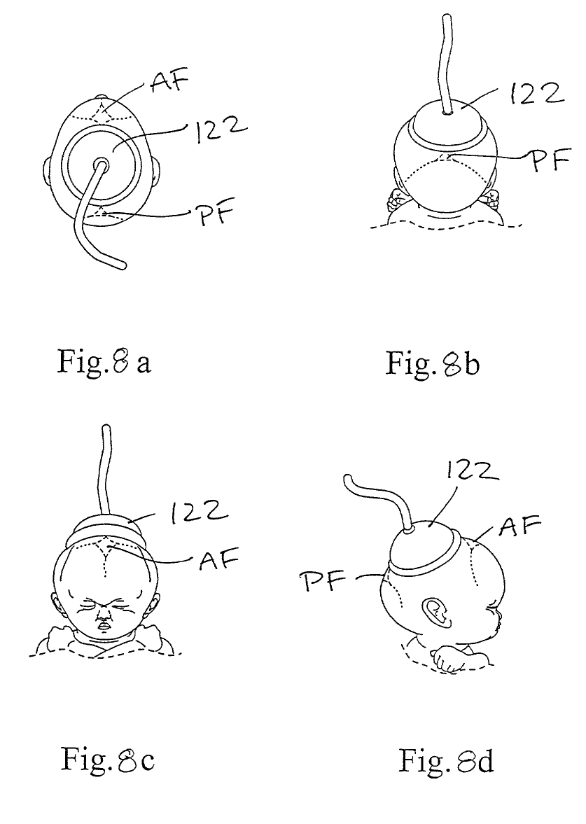

FIG. 5 shows an obstetrical imaging system 100 integrated with a vacuum extractor 120. FIG. 6 is an underside view of the integrated obstetrical imaging system 100 and vacuum extractor 120 of FIG. 5. The vacuum extractor 120 includes a vacuum-gripping cup 122, a vacuum pump 124 connected to the cup via a vacuum hose 126 and a pull handle 128 attached to the cup 122. The vacuum-gripping cup 122 may be flexible or rigid and is typically about 5 cm in diameter. In one particularly preferred embodiment, the vacuum-gripping cup 122 is rigid and has a flexible sealing ring 130 around the rim of the cup 122. The vacuum extractor 120 may be used with an electric vacuum pump or with a manually operated vacuum pump 124. Alternatively, the vacuum extractor 120 may be used with a hospital vacuum source. Optionally, the vacuum pump 124 may be integrated into the pull handle 128 of the device, as shown.

An obstetrical imaging system 100 is integrated into the vacuum extractor 120 to facilitate correct placement of the vacuum-gripping cup 122 on the head of the fetus. The obstetrical imaging system 100 includes a miniaturized video camera 102 mounted on the vacuum-gripping cup 122 and an illumination subsystem 104. The miniaturized video camera 102 may be permanently mounted on the vacuum-gripping cup 122 or it may be removable so that the same miniaturized video camera 102 can be used interchangeably with the vacuum extractor 120 and with the finger-mounted obstetrical imaging system 100 shown in FIGS. 1 and 2. A video cable 106 connects the miniaturized video camera 102 to a display monitor 108. The illumination subsystem 104 includes a light source 110 and a fiber optic cable 112 that is configured to distribute the illumination around the rim 132 of the vacuum-gripping cup 122. In one particularly preferred embodiment, one or more optical fibers 114 form a light-emitting ring 134 that encircles the rim 132 of the vacuum-gripping cup 122. The rim 132 of the vacuum-gripping cup 122 is shown in cross section in FIG. 7. In an exemplary embodiment, the light-emitting ring 143 is formed by stripping the cladding 136 away along one side of the optical fiber 114 so that light can escape through the side of the optical fiber 114. A flat face 138 or other lens-shaped surface may be ground into the side of the optical fiber 114 to form the escaping light into a directed beam. Preferably, the light-emitting ring 134 is shielded by the flexible sealing ring 130 so that substantially all of the light from the optical fibers 114 enters the tissue of the scalp and a minimum of light escapes. The light from the light-emitting ring 134 transilluminates the tissue of the scalp and makes the sutures, fontanelles and other structures beneath the scalp visible.

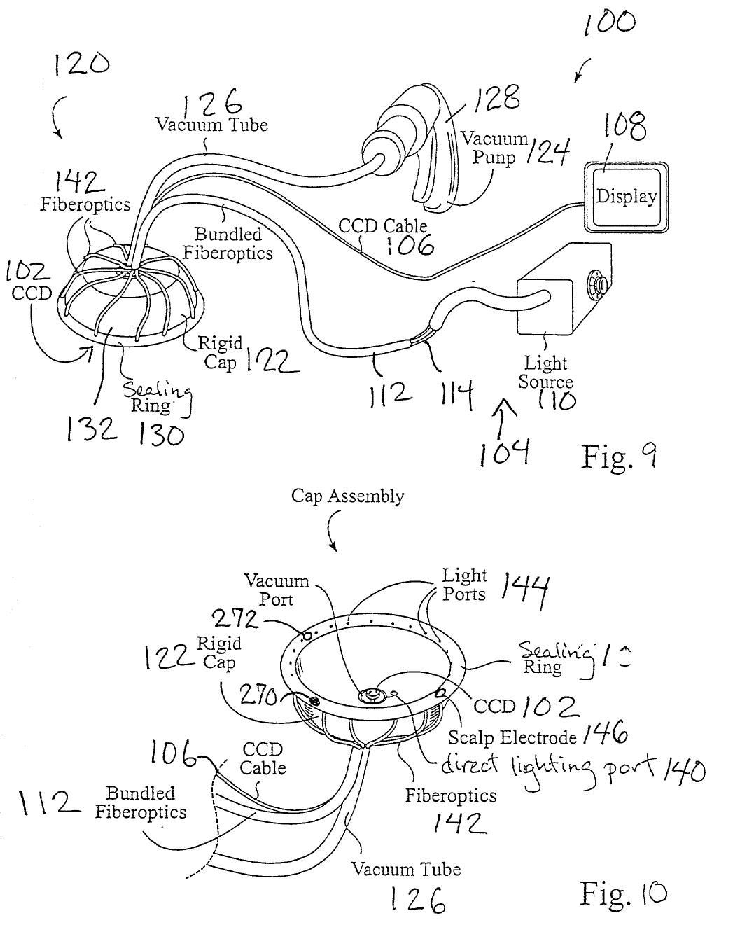

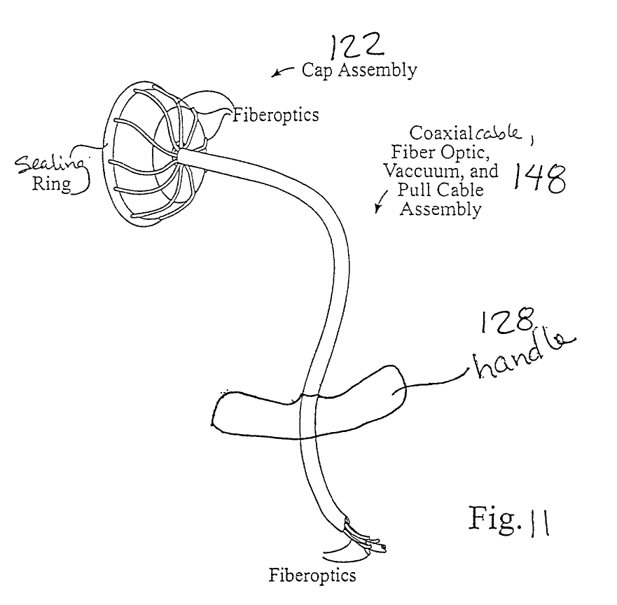

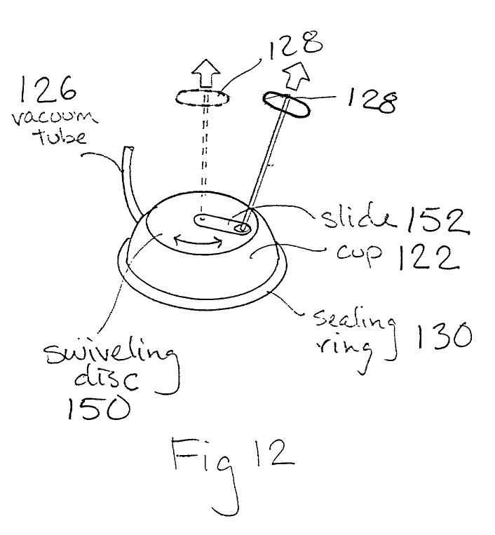

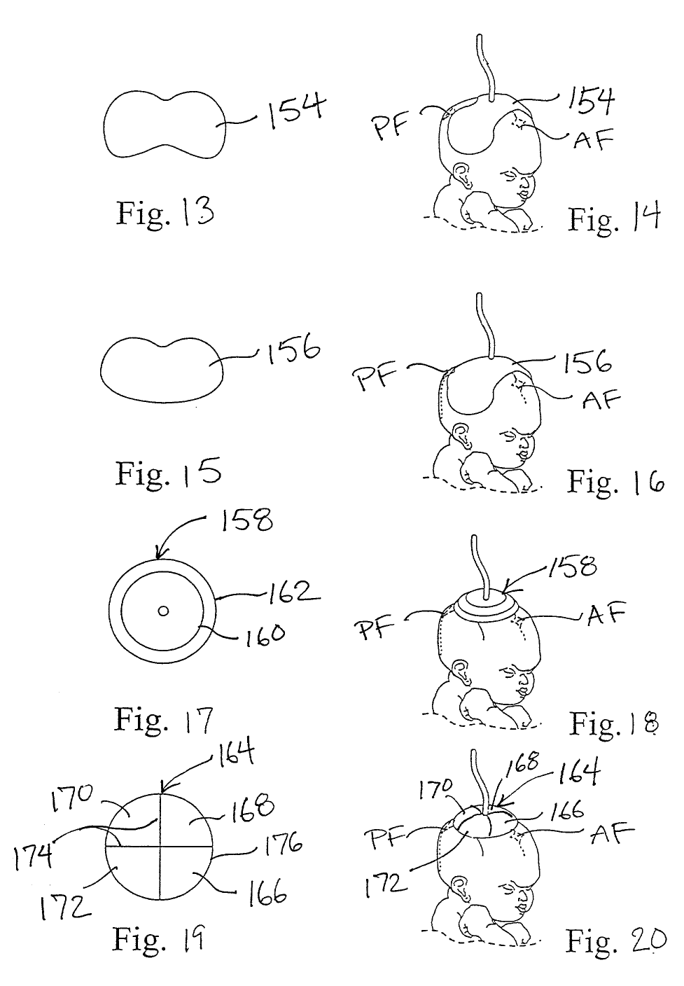

The miniaturized video camera 102, which is mounted facing distally within the vacuum-gripping cup 122, captures the image of the scalp beneath the cup 122 and displays it on a display monitor 108. By moving the vacuum-gripping cup 122, the obstetrician can get a full view of the fetal scalp and can identify the sutures, fontanelles and other structures on the head of the fetus to diagnose the position of the fetus and can accurately estimate the size of the fetus. Once the fontanelles have been identified, the vacuum-gripping cup 122 can be placed in the preferred target position between the fontanelles and a vacuum can be applied to grip the head. If either of the fontanelles is visible within the vacuum-gripping cup 122, a vacuum should not be applied until the vacuum-gripping cup 122 is correctly repositioned. FIGS. 8a-8d show the vacuum cup 122 of the integrated obstetrical imaging system and vacuum extractor of FIG. 5 placed on a fetus in the preferred target position between the anterior fontanelle AF and posterior fontanelle PF.

Optionally, the illumination subsystem may also include a direct lighting port 140 for illuminating the scalp beneath the vacuum-gripping cup 122 directly in order to view the external features of the scalp. Normally, light to the direct lighting port 140 would be turned off when viewing the internal structures by transillumination.

The finger-mounted obstetrical imaging system 100 of FIG. 1 can also be used to verify the correct placement of the vacuum-gripping cup 122. The finger-mounted obstetrical imaging system 100 can be used for direct visualization of the scalp or the light source 110 can be turned off in order to view the internal structures by transillumination.

Additional independent light sources may be used in conjunction with the imaging systems of FIG. 1 or 5 for direct illumination or transillumination of the fetal scalp.

FIG. 9 shows an alternate embodiment of the obstetrical imaging system 100 integrated with a vacuum extractor 120. FIG. 10 is an underside view of the integrated obstetrical imaging system 100 and vacuum extractor 120 of FIG. 9. This embodiment is similar to that shown in FIGS. 5-7 except that the illumination subsystem 104 includes multiple optical fibers 142 that are distributed around the rim 132 of the vacuum-gripping cup 122 assembly to form multiple light ports 144. Preferably, the light ports 144 are shielded by the flexible sealing ring 130 so that substantially all of the light from the optical fibers 142 enters the tissue of the scalp and a minimum of light escapes.

Alternatively, the illumination subsystem 104 may utilize one or more light emitting diodes (LED's) or laser diodes distributed around the rim 132 of the vacuum-gripping cup 122 as a light source for transillumination of the fetal scalp. In addition, light sensors located on the rim 132 of the vacuum-gripping cup 122 may be provided for measuring the absorption and/or reflectance of the light by the tissues of the fetal scalp. This information can be used to diagnose the fontanels and other structures of the fetal scalp and the position of the vacuum-gripping cup 122 with respect to those structures.

Alternatively or in addition, the obstetrical imaging system 100 may utilize one or more ultrasonic sensors distributed around the rim 132 of the vacuum-gripping cup 122. The ultrasonic sensors are capable of distinguishing between bone and soft tissue. Information from an array of ultrasonic sensors distributed around the rim 132 of the vacuum-gripping cup 122 will be displayed on a monitor. This information can be used to diagnose the fontanels and other structures of the fetal scalp and the position of the vacuum-gripping cup 122 with respect to those structures.

FIG. 10 shows another feature that may be combined with this embodiment or any other embodiment of the vacuum extractor 120. Optionally, the vacuum-gripping cup 122 assembly may include one or more scalp electrodes 146 located on the flexible sealing ring 130 around the rim 132 of the cup 122 for monitoring the fetal heartbeat during deliver. Once a vacuum is applied, the scalp electrodes 146 will make good electrical contact with the scalp tissue without the need to pierce the skin with the electrode 146. The sensing circuit for monitoring the fetal heartbeat can be completed with a second electrode 272, which may be placed externally on the mother, elsewhere on the fetus or elsewhere on the rim 132 of the vacuum-gripping cup 122. This feature allows the fetal heartbeat to be monitored continuously while the vacuum extractor 120 is in use, unlike typical prior art scalp electrodes, which must be removed before applying a vacuum extractor.

Another feature shown in FIG. 10 may also be combined with this embodiment or any other embodiment of the vacuum extractor 120. Optionally, the vacuum-gripping cup 122 assembly may include one or more location marking devices 270, preferably positioned near the rim 132 of the cup 122 so that they are in close proximity to the scalp of the fetus. The location marking device 270 may be passive, e.g. a magnet, or active, e.g. a low power radio transmitter. A location monitoring device is mounted externally on the patient to monitor the position of the location marking device 270 in order to track the progress of the fetus as it descends through the birth canal. Location monitoring devices suitable for use in combination with the vacuum extractor 120 of the present invention are described in U.S. Pat. Nos. 5,935,061, 6,200,279 and 6,270,458, which are hereby incorporated by reference in their entirety. The information provided by the location monitoring device can be used to determine the treatment course for delivery of the fetus. For example, if no progress is detected, the obstetrician may recommend cesarean delivery, particularly if the heart rate monitor or other parameters indicate fetal stress.

FIG. 11 shows another alternate embodiment of the integrated obstetrical imaging system and vacuum extractor. This embodiment is similar to that shown in FIGS. 9-10 except that the video cable, fiber optics, vacuum hose and pull cable are integrated into a single cable assembly 148 with a pull handle 128 attached.

FIG. 12 shows an alternate handle assembly for an integrated obstetrical imaging system and vacuum extractor. In this embodiment, the pull handle 128 is pivotally attached to a swiveling disc 150 in the base of the vacuum-gripping cup 122. The swiveling disc 150 allows the pull handle 128 to be pivoted with respect to the vacuum-gripping cup 122 so that tension can be applied at any desired vector angle. In addition, the pull handle 128 may be attached to a radial slide 152 on the swiveling disc 150 that allows the point of attachment for the handle 128 to be selectively placed near the edge of the vacuum-gripping cup 122, as shown in solid lines, or near the center of the vacuum-gripping cup, as shown in phantom lines.

FIG. 13 shows an alternate configuration of a vacuum-gripping cup 154 for a vacuum extractor. In this embodiment, the vacuum-gripping cup 154 is shaped in a butterfly configuration to provide increased surface area for gripping the head of the fetus without covering the anterior AF or posterior fontanelle PFs. The vacuum-gripping cup 154 may be flexible or rigid and may be constructed with or without a flexible sealing gasket around the rim. The vacuum-gripping cup 154 may be integrated with an obstetrical imaging system as described above or it may be used independently. FIG. 14 shows the vacuum-gripping cup 154 of FIG. 13 placed on a fetus in the preferred target position.

FIG. 15 shows an alternate configuration of a vacuum-gripping cup 156 for a vacuum extractor. In this embodiment, the vacuum-gripping cup 156 is constructed in a kidney-shaped configuration to provide increased surface area for gripping the head of the fetus without covering the anterior or posterior fontanelles. The vacuum-gripping cup 156 may be flexible or rigid and may be constructed with or without a flexible sealing gasket around the rim. The vacuum-gripping cup 156 may be integrated with an obstetrical imaging system as described above or it may be used independently. FIG. 16 shows the vacuum-gripping cup 156 of FIG. 15 placed on a fetus in the preferred target position.

FIG. 17 shows an alternate configuration of a vacuum-gripping cup 158 for a vacuum extractor. In this embodiment, the vacuum-gripping cup 158 is constructed with two or more concentric vacuum chambers 160, 162 that are independently controllable. The vacuum-gripping cup 158 may be flexible or rigid with flexible sealing gaskets around the rims of the concentric vacuum chambers 160, 162. The vacuum-gripping cup 158 may be integrated with an obstetrical imaging system as described above or it may be used independently. FIG. 18 shows the vacuum-gripping cup 158 of FIG. 17 placed on a fetus in the preferred target position. Normally, the vacuum-gripping cup 158 would be placed to avoid the anterior fontanelle AF and posterior fontanelle PF. However, if this preferred placement was not possible or practical to achieve, the vacuum-gripping cup 158 may be placed over one or both of the fontanelles. In this case, a high vacuum can be safely applied in the center vacuum chamber 160 which has been placed to avoid the fontanelles, but a lower vacuum or no vacuum should be applied in the outer vacuum chamber 162 to avoid injury to the fontanelles.

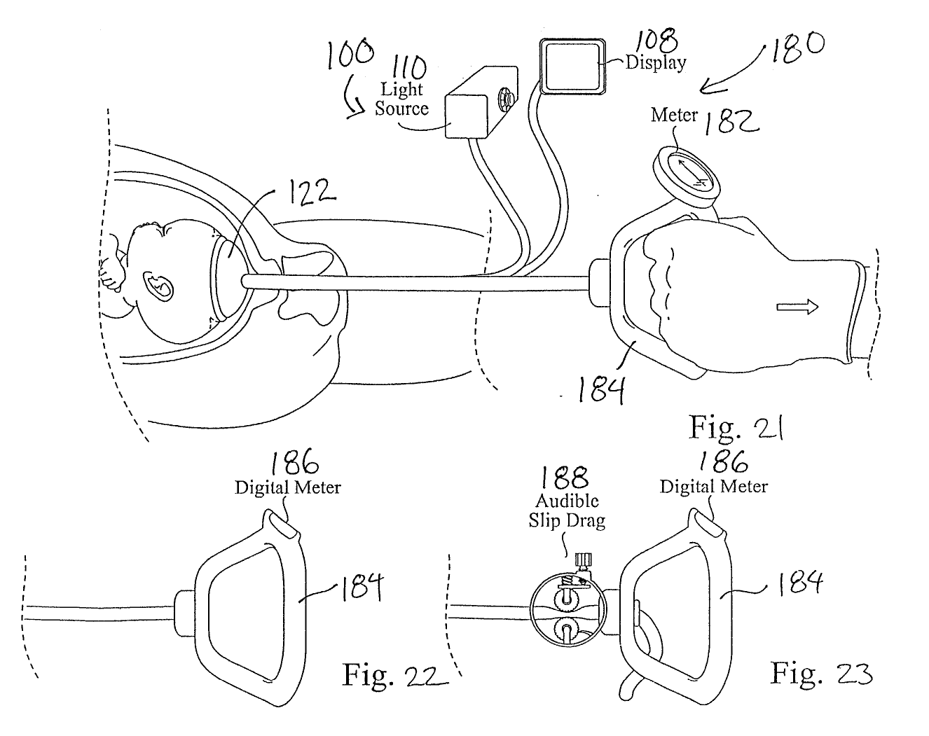

FIG. 19 shows another alternate configuration of a vacuum-gripping cup 164 for a vacuum extractor. In this embodiment, the vacuum-gripping cup 164 is constructed with multiple sector-shaped vacuum chambers 166, 168, 170, 172 that are independently controllable. The vacuum-gripping cup 164 may be flexible or rigid with flexible sealing gaskets around the rim 176 and along the walls 174 separating the vacuum chambers 166, 168, 170, 172. The vacuum-gripping cup 164 may be integrated with an obstetrical imaging system as described above or it may be used independently. FIG. 20 shows the vacuum extractor 164 of FIG. 19 placed on a fetus in the preferred target position. Normally, the vacuum-gripping cup 164 would be placed to avoid the anterior fontanelle AF and posterior fontanelle PF. However, if this preferred placement was not possible or practical to achieve, the vacuum-gripping cup 164 may be placed over one or both of the fontanelles. In this case, a high vacuum can be safely applied in the vacuum chambers 168, 172 which have been placed to avoid the fontanelles, but a lower vacuum or no vacuum should be applied in any vacuum chambers 166, 170 that cover the fontanelles to avoid injury.

FIG. 21 shows a vacuum extractor 180 with an analog tension meter 182 integrated into the pull handle 184. FIG. 22 shows an alternate embodiment of the vacuum extractor handle 184 with an integral digital tension meter 186. The pull handle 184 with the integral tension meter 182 may be integrated with an obstetrical imaging system 100 as shown or it may be used without the imaging system. The integral tension meter 182 allows the obstetrician to monitor the tension that is applied to the vacuum extractor 180 in order to avoid injury to the fetus and to avoid premature release of the vacuum-gripping cup 122. Optionally, the pull handle 184 may also include an audible alarm to notify the obstetrician when a predetermined tension level has been reached or exceeded.

FIG. 23 shows a vacuum extractor handle 184 with an adjustable tension drag 188. The tension drag 188 can be adjusted to limit the tension applied to the vacuum extractor to a predetermined maximum, beyond which the tension drag 188 will slip to relieve any excess tension on the handle 184 in order to avoid injury to the fetus and to avoid premature release of the vacuum-gripping cup 122. Alternatively or in addition, the tension drag 188 may be configured with a factory-set maximum tension. Optionally, the tension drag 188 may be configured to create an audible noise when the tension drag 188 slips to indicate to the obstetrician when a predetermined tension level has been reached or exceeded. The pull handle 184 with adjustable tension drag 188 may be integrated with an obstetrical imaging system as described above or it may be used without the imaging system.

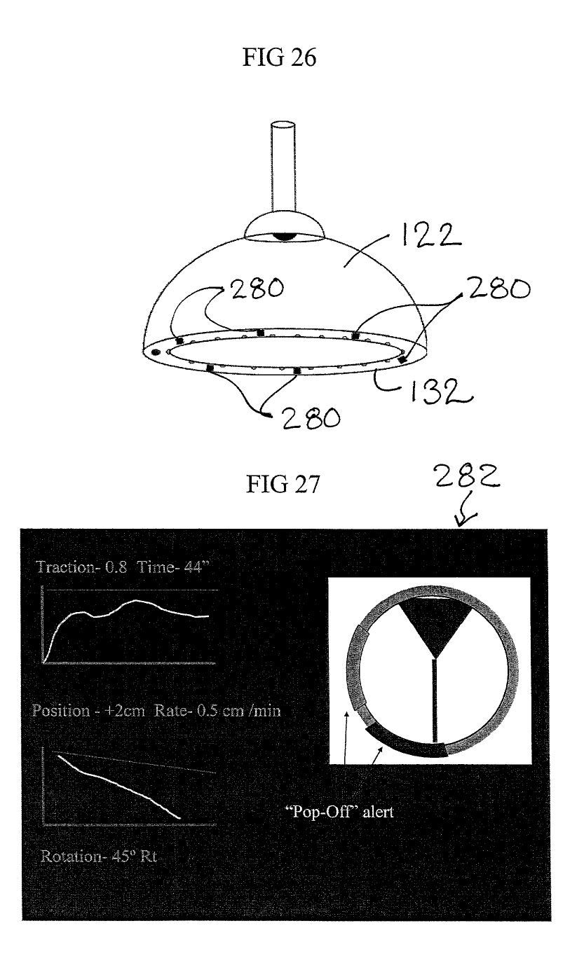

Alternately or in addition, as shown in FIG. 26, the rim 132 of the vacuum-gripping cup 122 may be instrumented with one or more contact or pressure sensors 280 for sensing impending detachment of the vacuum-gripping cup 122 from the scalp of the fetus. The correct vector of the traction is important for the success of the procedure and detachment of the rim 132 of the vacuum-gripping cup 122 in one area causes detachment of the whole vacuum-gripping cup 122. The array of sensors 280 will indicate which portions of the rim 132 are about to detach and the system will display this information on a monitor screen 282, as shown in FIG. 27, and alert the operator so that adjustments can be made to the force and/or direction of the traction vector being applied to avoid premature release of the vacuum-gripping cup 122 and to provide the best chances of a successful fetal extraction.

Another safety mechanism that can be integrated into a vacuum extractor is a safety shutoff valve for the vacuum source, particularly when the vacuum extractor is used with an electric vacuum pump or with a hospital vacuum source. The safety shutoff valve may be triggered by a sudden drop in vacuum in the vacuum-gripping cup 122 and/or by a sudden drop in the tension on the handle 184, either of which may indicate premature release of the vacuum-gripping cup 122. The safety shutoff valve prevents the vacuum-gripping cup 122 from inadvertently reattaching to the fetus in an undesired position or to the mother's tissues after a premature release of the vacuum-gripping cup 122.

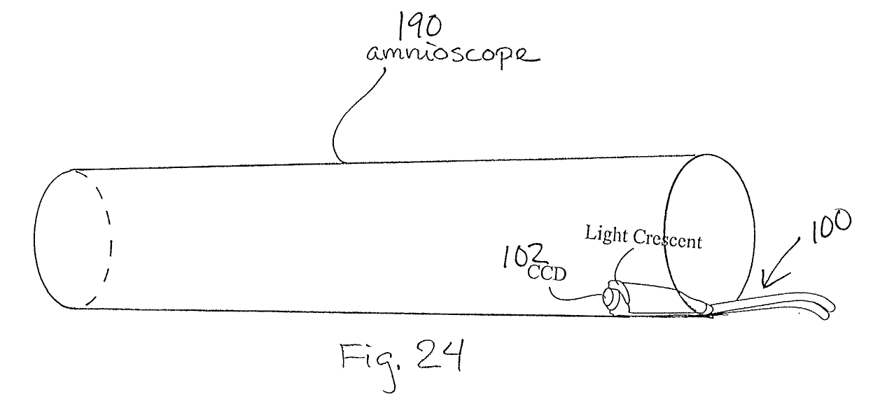

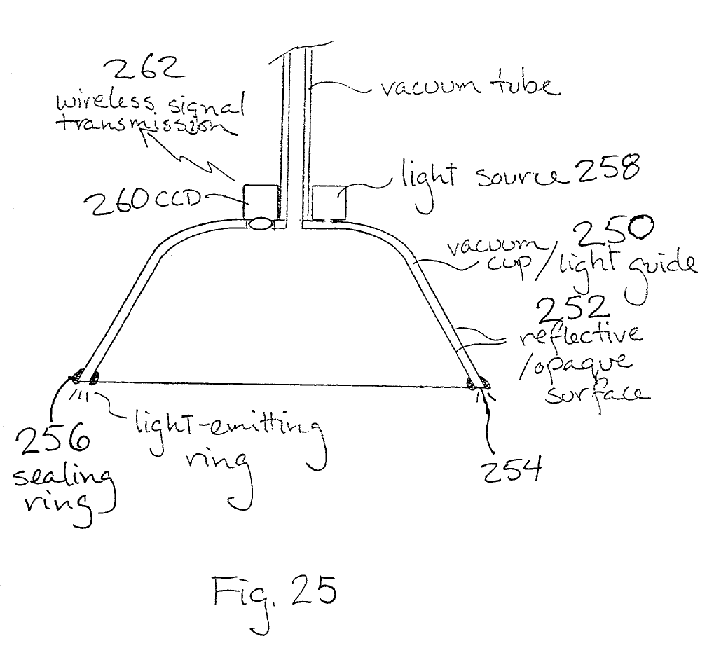

FIG. 24 shows a miniaturized obstetrical imaging system 100 integrated with a transcervical amnioscope 190. The amnioscope 190 is a typically a tapered metal or plastic tube that is inserted through the mother's cervical canal to provide a direct view of the fetus through the intact membranes. The amnioscope 190 is used for diagnosis of meconium in the amniotic fluid, placenta previa and other conditions and for insertion of other instruments, such as a scalpel and capillary tube for taking a fetal blood sample from the scalp. The direct imaging can be augmented with videoendoscopic imaging by mounting a miniaturized obstetrical imaging system 100, similar to that described in FIGS. 1 and 2 within the lumen of the amnioscope 190. The miniaturized video camera 102 may be permanently mounted on the amnioscope 190 or it may be removable so that the same miniaturized video camera 102 can be used interchangeably with the amnioscope 190 of FIG. 24, the vacuum extractor of FIGS. 5-7 and with the finger-mounted obstetrical imaging system 100 of FIGS. 1 and 2. The small size of the miniaturized video camera 102 does not interfere with direct visualization or with access for other instruments.

Alternatively, a miniaturized video camera 102 can be mounted on a mandrel for insertion into a standard tubular transcervical amnioscope 190.

FIG. 25 shows another alternate embodiment of the obstetrical imaging system integrated with a vacuum extractor in cross section. This embodiment demonstrates a number of features that may be used separately or in combination with other features described in connection with other embodiments of the obstetrical imaging system. The vacuum-gripping cup 250 shown in FIG. 25 is preferably molded of an optically transparent polymer material, such as polymethyl methacrylate (PMMA) or polycarbonate, to serve as a light guide. The vacuum-gripping cup 250 may be coated on its inner and/or outer surface with a reflective and/or opaque coating 252. The rim 254 of the vacuum-gripping cup 250 is uncoated so that it serves as a light-emitting ring. Preferably, the rim 254 of the vacuum-gripping cup 250 also includes a flexible sealing ring 256. The optically transparent vacuum-gripping cup 250 is coupled to a light source 258, shown in this embodiment as a compact, battery powered light source 258. The light from the light source 258 is transmitted by internal reflection through the optically transparent vacuum-gripping cup 250 to the light-emitting ring on the rim 254 of the cup 250. Optionally, the miniaturized video camera 260 may be a self-contained, battery powered CCD camera 260 with a wireless signal transmitter 262 to transmit the image signals to a display monitor.

Each of the various embodiments of the obstetrical imaging system may be configured for three-dimensional stereoscopic imaging. In addition, the obstetrical imaging system may be configured to include a sonic or ultrasonic range finder to estimate the distance to the target and to adjust the focus of the camera accordingly. In the case of three-dimensional stereoscopic imaging, the signal from the range finder may also be used to adjust the stereoscopic convergence angle of the imaging system.

Each of the various embodiments of the obstetrical imaging system may also be configured to include a selective wavelength and/or multiple wavelength light source to provide optimum illumination for visualizing different features of the external and/or internal anatomy of the fetal head. Additionally, means may be provided for clearing the lens and/or light source of the obstetrical imaging system in case it becomes blocked or contaminated in use. Possible mechanisms for clearing the lens and/or light source include a mechanical wiper, a visor, a fluid spray nozzle and/or a vacuum port. Alternatively or in addition, a mechanism can be included that spins the lens and/or a protective cover over the lens quickly so that it sheds any secretions or debris that may have collected on it.

Each of the various embodiments of the obstetrical imaging system may be configured as a piece of durable equipment to be resterilized and reused. Alternatively, some or all of the obstetrical imaging system may be configured as a disposable product for one-time use only.

Additionally, the obstetrical imaging system may be connected either by wiring or in a wireless fashion to an analyzing computerized system that will receive the imaging signal or integrate multiple pictures into a computerized digital image. The system will record and store the data, and will be able to provide parameters such as the level of tension and pressure applied and the time of the total procedure. This data will be transferred to a storage media and/or be printed. The system may also be adapted to display and/or record additional data, such as fetal heart rate, fetal blood pH, etc.

The system will be able to calculate the fetal weight by measuring the distance between the fontanels by the imaging system. This distance will be plotted against a known normogram of fetal weight and inter-fontanellar distance.

In a variation of the method according to the present invention, the integrated obstetrical imaging system and vacuum extractor described herein can be used to diagnose the position of the head of a fetus during spontaneous vaginal delivery using tissue transillumination or any of the other imaging modalities described above. The system can be used to monitor and document the progress of the delivery. If the delivery progresses normally and no problems such as malpositioning of the fetus are detected, vacuum extraction would not be applied. A simplified version of the invention may be used in this method to diagnose the position of the head of a fetus during spontaneous vaginal delivery without vacuum extraction. Such a device would include the illumination and imaging subsystems of the invention as described above, and optionally may also include one or more scalp electrodes for heart rate monitoring and/or location marking devices, but, for simplicity, it could be constructed without any vacuum connection or other features related to the vacuum extraction technique.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof. For example, many of the features described can be used together in combinations other than those explicitly described.

What is claimed is:

1. An obstetrical imaging system, comprising:
an optical imaging system configured to view a presenting part of a fetus during delivery;
an illumination subsystem configured to direct light through tissue within the visual field of the optical imaging system; and
a display monitor for displaying images from the optical imaging system.

2. The obstetrical imaging system of claim 1, wherein the optical imaging system comprises a miniaturized video camera.

3. The obstetrical imaging system of claim 1, wherein the optical imaging system is mounted on a cup-shaped member configured to contact a scalp of the fetus, and wherein the illumination subsystem is configured to emit light from a distal rim of the cup-shaped member.

4. An obstetrical imaging and fetal extraction system, comprising:
a vacuum-gripping cup configured for connection to a vacuum source;
an optical imaging system configured to view an area distal to the vacuum-gripping cup; and
an illumination subsystem configured to provide light in the visual field of the optical imaging system.

5. The obstetrical imaging and fetal extraction system of claim 4, further comprising a system for recording an image of a position of the vacuum-gripping cup relative to a head of a fetus.

6. The obstetrical imaging and fetal extraction system of claim 4, wherein the optical imaging system comprises a miniaturized video camera.

7. The obstetrical imaging and fetal extraction system of claim 4, wherein the illumination subsystem is configured to direct light through tissue within the visual field of the optical imaging system.

8. The obstetrical imaging and fetal extraction system of claim 7, wherein the illumination subsystem is configured to emit light from a distal rim of the vacuum-gripping cup.

9. The obstetrical imaging and fetal extraction system of claim 4, wherein the optical imaging system is configured to view an area within the interior of the vacuum-gripping cup.

10. The obstetrical imaging and fetal extraction system of claim 4, further comprising a fetal scalp electrode mounted on a distal rim of the vacuum-gripping cup.

11. The obstetrical imaging and fetal extraction system of claim 4, further comprising a tension meter for measuring tension applied to the vacuum-gripping cup.

12. The obstetrical imaging and fetal extraction system of claim 11, further comprising an audible alarm for indicating when a predetermined level of tension has been reached.

13. The obstetrical imaging and fetal extraction system of claim 5, further comprising an adjustable drag mechanism for limiting tension applied to the vacuum-gripping cup.

14. The obstetrical imaging and fetal extraction system of claim 5, wherein the vacuum-gripping cup is configured with a plurality of vacuum chambers with independently controllable vacuum levels.

15. An obstetrical fetal extraction system, comprising:
a vacuum-gripping cup configured for connection to a vacuum source; and
a fetal scalp electrode mounted on a distal rim of the vacuum-gripping cup.

16. The obstetrical fetal extraction system of claim 15, further comprising a second fetal scalp electrode mounted on the distal rim of the vacuum-gripping cup.

17. The obstetrical imaging and fetal extraction system of claim 4, further comprising means for monitoring and recording descent of a fetus within a birth canal.

18. An obstetrical fetal extraction system, comprising:
a vacuum-gripping cup configured for connection to a vacuum source; and
a tension meter for measuring tension applied to the vacuum-gripping cup.

19. The obstetrical fetal extraction system of claim 18, further comprising an audible alarm for indicating when a predetermined level of tension has been reached.

20. The obstetrical fetal extraction system of claim 18, further comprising an adjustable drag mechanism for limiting tension applied to the vacuum-gripping cup.

21. The obstetrical fetal extraction system of claim 18, further comprising a system for recording and displaying progress of a fetal extraction procedure, including a graph of tension applied to the vacuum-gripping cup versus time.
